(19) United States
(12) Patent Application Publication
(10) Pub. No.: US 2021/0137100 A1
(43) Pub. Date: May 13, 2021

(54) AN APPARATUS TO MAINTAIN TEMPERATURE OF AN OBJECT

(71) Applicant: INDIAN INSTITUTE OF SCIENCE, Bangalore (IN)

(72) Inventors: Balan GURUMOORTHY, Bangalore (IN); Ashitava GHOSAL, Bangalore (IN); Rohit S NAMBIAR, Bangalore (IN); Deval KARIA, Bangalore (IN)

(73) Assignee: INDIAN INSTITUTE OF SCIENCE, Bangalore (IN)

(21) Appl. No.: 17/048,152

(22) PCT Filed: Apr. 16, 2019
(86) PCT No.: PCT/IB2019/053114

§ 371 Date: Oct. 16, 2020

(30) Foreign Application Priority Data
Apr. 17, 2018 (IN) 201841006081

(51) Int. Cl. A01N 1/02

(52) U.S. Cl. CPC A01N 1/0252; A61B 10/0096

(57) ABSTRACT

The present disclosure provides a portable apparatus to maintain temperature of an object comprising a first chamber containing a quantity of a thermal material. The first chamber is thermally coupled to a container holding the object of interest. Ambient air is driven over the thermal material and then towards the container to change the temperature of the container. A valve is provided near the container which can be opened to allow entry of ambient air towards the container. Either of the heated or cooled air flow and the operation of the valve is regulated to maintain the temperature of the container to within a predetermined temperature range. The container is configured to be sealed to isolate the object from the ambient and allows for a means to preserve object quality. A housing of the apparatus is provided with a provision to protect the first container from mechanical shocks.

### TECHNICAL FIELD

present disclosure relates generally to the field of apparatuses to handle sample objects. In particular, the present disclosure pertains to a portable, energy efficient apparatus to maintain the temperature of an object.

### BACKGROUND

Background description includes information that may be useful in understanding the present invention. It is not an admission that any of the information provided herein is prior art or relevant to the presently claimed invention, or that any publication specifically or implicitly referenced is prior art.

In both commercial and domestic applications, maintaining integrity of objects that are prone to quick degradation often involves isolation of the object from the oxidation effects of the ambient and maintaining of the temperature of the object to retain the health of the object. It is, further, advantageous to have an apparatus for the same that is portable in order than the sample can be transported from one point to another if the case needs be.

Particularly, portable refrigeration is acquiring a wide expanse of applications both in domestic and commercial applications. One important sphere where portable refrigeration is critical is in the transport of materials that will degrade at ambient temperatures, particularly in pharmaceutical and bio-medical applications, such as drug and organ transport. Typically, in such applications, there is a need for not just cooling, but a need for maintaining temperature in a specific range and actively regulating the sample quality.

Portable refrigeration apparatuses available in the art rely on thermoelectric cooling technologies. While the technology is mature, it demands high-power consumption and a heavy setup. This precludes such systems from being viable in applications such as drone flights.

Using refrigerants such as dry ice is an option for developing apparatuses that are more energy efficient. Dry ice sublimates at 194.65 K (−78° C.; −109.3° F.) at atmospheric pressure. This significantly reduces and, in some cases, eliminates the need of electrical refrigerating systems. This is particularly useful in the case of drug, vaccine and organ transports, as the duration for which they remain viable in normothermic temperatures is quite short. However, even with dry ice, there is still a need for temperature regulation, since −78.5° C. is significantly lower than the permissible temperature range in which such temperature sensitive biological samples should be stored in.

There is, therefore, a requirement in the art for a portable refrigeration system that is light weight and energy efficient, which can further be implemented in applications such as drone-based transport of materials.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities or dimensions of items, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

### OBJECTS

A general object of the present disclosure is to provide a portable apparatus to maintain temperature of an object.

Another object of the present disclosure is to provide an apparatus that can maintain temperature of an object within a predetermined range.

Another object of the present disclosure is to provide an apparatus that is energy efficient.

Another object of the present disclosure is to provide a refrigeration and preservation apparatus for organ transport.

Another object of the present disclosure is to provide an apparatus with structural integrity to withstand nominal loads due to impact.

Another object of the present disclosure is to provide an apparatus with optimized design with respect to weight, size and dimensions.

### SUMMARY

The present disclosure relates generally to the field of apparatuses to handle sample objects. In particular, the present disclosure pertains to a portable, energy efficient apparatus to maintain the temperature of an object.

In an aspect, the present disclosure provides an apparatus to maintain temperature of a biological object, said apparatus comprising: a thermally insulated housing comprising: at least one first chamber configured to hold a first container, said first container adapted to hold a refrigerant; a second chamber thermally coupled to the at least one first chamber and adapted to hold a second container, said second container atmospherically isolated from the second chamber; a first fan coupled with the at least one first container and configured to induce flow of air at a first temperature over the refrigerant and into the second chamber, said first temperature being higher than a predetermined range of temperature for the biological object; a valve provided in the second chamber and operable to open to a source of air at a second temperature, said second temperature being higher than the predetermined range of temperature for the biological object; and a control unit configured to sense temperature inside the second container from one or more first temperature sensors provided inside the second container, wherein the control unit is configured to operate any of the first fan and the valve to enable the predetermined temperature range to be maintained inside the second container.

In an embodiment, the refrigerant is dry ice.

In another embodiment, one or more second temperature sensors are provided in the second chamber, wherein the control unit receives temperature input from the one or more second temperature sensors along with temperature input from the one or more first sensors to enable determination of correlation between temperature in the second chamber and the temperature inside the second container.

In another embodiment, the control unit comprises a microcontroller configured to receive input from the one or more first sensors and the one or more second sensors and operate the first fan, the valve and the second fan to enable the predetermined temperature range to be maintained inside the second container.

In another embodiment, a reservoir configured to store a preservation solution is provided in the second chamber, wherein a pump is configured to circulate the preservation solution to the biological object.

In another embodiment, a pressure sensor is configured to measure flow pressure of the preservation solution, and wherein the measured flow pressure value is provided as input to the control unit to enable the control unit to operate the pump to provide flow of the preservation solution that is within a predetermined range of pressure.

In another embodiment, a pH sensor is provided at a location so as to enable the sensor to measure pH of the preservation solution after the preservation solution has interacted with the biological object, and wherein the measured pH is provided as input to the control unit to enable the control unit to operate the pump to provide optimal flow of the preservation solution based on the measured pH of the used preservation solution.

In another embodiment, a drain is provided to receive used preservation solution.

In another embodiment, a second fan is provided to induce flow of ambient through the valve when the valve is open.

In another embodiment, the second container is made of a thermally conducting material.

In another embodiment, the walls of the housing comprise one or more elements configured to absorb physical shock, said one or more elements selected from a group comprising springs, lattice structures and elastomer layers.

In another embodiment, the apparatus is provided with a control unit that is configurable.

In an aspect the present disclosure provides an apparatus to maintain temperature of an object, said apparatus comprising: a thermally insulated housing comprising: at least one first chamber configured to hold a first container, said first container adapted to hold a thermal material; a second chamber thermally coupled to the at least one first chamber and adapted to hold a second container, said second container atmospherically isolated from the second chamber; a first fan coupled with the at least one first container and configured to induce flow of air at a first temperature over the thermal material and into the second chamber, said first temperature being outside of a first limit of a predetermined range of temperature for the biological object; a valve provided in the second chamber and operable to open to a source of air at a second temperature, said second temperature being outside of the first limit of the predetermined range of temperature for the biological object; and a control unit configured to sense temperature inside the second container from one or more first temperature sensors provided inside the second container, wherein the control unit is configured to operate any of the first fan and the valve to enable the predetermined temperature range to be maintained inside the second container.

In an embodiment, the thermal material is a refrigerant.

In another embodiment, the thermal material is a heating material.

In another embodiment, one or more second temperature sensors are provided in the second chamber, wherein the control unit receives temperature input from the one or more second temperature sensors along with temperature input from the one or more first sensors to enable determination of correlation between temperature in the second chamber and the temperature inside the second container.

In another embodiment, the control unit comprises a microcontroller configured to receive input from the one or more first sensors and the one or more second sensors and operate the first fan, the valve and the second fan to enable the predetermined temperature range to be maintained inside the second container.

Various objects, features, aspects and advantages of the inventive subject matter will become more apparent from the following detailed description of preferred embodiments, along with the accompanying drawing figures in which like numerals represent like components.

### BRIEF DESCRIPTION OF DRAWINGS

The accompanying drawings are included to provide a further understanding of the present invention and are incorporated in and constitute a part of this specification. The drawings illustrate exemplary embodiments of the present invention and, together with the description, serve to explain the principles of the present invention.

FIG. 1 illustrates a schematic representation of an apparatus to maintain temperature of an object, in accordance with an embodiment, of the present disclosure.

FIG. 2 illustrates a schematic representation of a cross-sectional view of the proposed apparatus to maintain temperature of an object, in accordance with an embodiment of the present disclosure.

FIG. 3 illustrates a schematic representation of an exemplary valve for the second vent of the proposed apparatus, in accordance with an embodiment of the present disclosure.

FIGS. 4A and 4B illustrate schematic representations of the housing of the proposed apparatus, in accordance with an embodiment of the present disclosure.

FIG. 5 illustrates an exemplary representation of the proposed apparatus with a display and control panel, in accordance with an embodiment of the present disclosure.

FIG. 6 illustrates an exemplary representation of a microcontroller-based system to operate the proposed refrigeration apparatus, in accordance with an embodiment of the present disclosure.

FIG. 7 illustrates an exemplary representation of a multi-function refrigeration apparatus, in accordance with an embodiment of the present disclosure.

FIG. 8 illustrates an exemplary representation of the refrigerant container in the proposed multi-function refrigeration apparatus, in accordance with an embodiment of the present disclosure.

FIG. 9 illustrates an exemplary representation of the organ container in the proposed multi-function refrigeration apparatus, in accordance with an embodiment of the present disclosure.

FIG. 10 illustrates an exemplary plot of temperatures of second chamber and heart container with time for the proposed multi-function refrigeration apparatus, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Exemplary embodiments will now be described more fully hereinafter with reference to the accompanying drawings, in which exemplary embodiments are shown. These exemplary embodiments are provided only for illustrative purposes and so that this disclosure will be thorough and complete and will fully convey the scope of the invention to those of ordinary skill in the art. The invention disclosed may, however, be embodied in many different forms and should not be construed as limited to the embodiments set forth herein. Various modifications will be readily apparent to persons skilled in the art. The general principles defined herein may be applied to other embodiments and applications without departing from the spirit and scope of the invention. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

In an aspect, the present disclosure provides an apparatus to maintain temperature of a biological object, said apparatus comprising: a thermally insulated housing comprising: at least one first chamber configured to hold a first container, said first container adapted to hold a refrigerant; a second chamber thermally coupled to the at least one first chamber and adapted to hold a second container, said second container atmospherically isolated from the second chamber; a first fan coupled with the at least one first container and configured to induce flow of air at a first temperature over the refrigerant and into the second chamber, said first temperature being higher than a predetermined range of temperature for the biological object; a valve provided in the second chamber and operable to open to a source of air at a second temperature, said second temperature being higher than the predetermined range of temperature for the biological object; and a control unit configured to sense temperature inside the second container from one or more first temperature sensors provided inside the second container, wherein the control unit is configured to operate any of the first fan and the valve to enable the predetermined temperature range to be maintained inside the second container.

In an aspect the present disclosure provides an apparatus to maintain temperature of an object, said apparatus comprising: a thermally insulated housing comprising: at least one first chamber configured to hold a first container, said first container adapted to hold a thermal material; a second chamber thermally coupled to the at least one first chamber and adapted to hold a second container, said second container atmospherically isolated from the second chamber; a first fan coupled with the at least one first container and configured to induce flow of air at a first temperature over the thermal material and into the second chamber, said first temperature being outside of a first limit of a predetermined range of temperature for the biological object; a valve provided in the second chamber and operable to open to a source of air at a second temperature, said second temperature being outside of the first limit of the predetermined range of temperature for the biological object; and a control unit configured to sense temperature inside the second container from one or more first temperature sensors provided inside the second container, wherein the control unit is configured to operate any of the first fan and the valve to enable the predetermined temperature range to be maintained inside the second container.

FIG. 1 illustrates a schematic representation of an apparatus to maintain temperature of an object, in accordance with an embodiment, of the present disclosure. In an embodiment, the apparatus 100 comprises a housing 102 with one or more chambers. A first container 104 with one or more vents 106 that is configured to store a quantity of thermal material 108 is adapted to fit into a first chamber 110. In an exemplary embodiment, the thermal material 108 can be a cooling material or a heating material. The first container 104 is further coupled with a first fan 112 that allows for flow of air at a first temperature through the first container 104 in order that the air exchanges heat with the thermal material 108.

In another embodiment, a second container 114 is adapted to fit into a second chamber 116. The second container 114 can be made of a thermally conducting material such as stainless steel and can be configured to hold an object whose temperature is to be maintained, such that once the second container 114 is closed, the object is completely isolated.

In an exemplary implementation, the air at the first temperature can be from the ambient or from another chamber of the one or more chambers.

In another embodiment, the second chamber 116 can be thermally coupled to the first chamber 110. The thermal coupling can be in the form of one or more vents between the first chamber 110 and the second chamber 116 corresponding to the one or more vents 106 in the first container 104. In another embodiment, the housing 102 can be sealed so as to be thermally isolated from the ambient conditions.

In another embodiment, the second container 114 is equipped with a temperature sensor T1 118 to detect temperature within the second container 114. The second chamber 116 is equipped with a temperature sensor T2 120 to detect temperature in the second chamber 116.

In another embodiment, the second chamber 116 also comprises a vent 122 to the ambient. The vent 122 is provided with a second fan 124 to allow flow of air at a second temperature into the second chamber 114. The vent 122 is accessible through a valve 126 that can be operated by an actuator 128. electronically operated. In an exemplary embodiment, the valve 126 can be operated by a servo motor.

In an exemplary implementation, the air at the second temperature can be from the ambient or from another chamber of the one or more chambers.

FIG. 2 illustrates a schematic representation of a cross-sectional view of the proposed apparatus to maintain temperature of an object, in accordance with an embodiment of the present disclosure. In an embodiment, the first fan 112 induces flow of air at the first temperature into the first container 104 and over the thermal material 108, thereby allowing heat exchange between the thermal material 108 and the air to change the temperature of the air to a second temperature. The force from the first fan 112 further forces the air at the second temperature to flow into the second chamber 114, thereby altering the temperature of the second chamber 116, and consequently, of the second container 114.

In another embodiment, temperatures of the second container 114 and the second chamber 116 are detected by the sensors T1 118 and T2 120 respectively. The temperatures are fed to a control unit which is configured to monitor the temperature. The control unit, which can be a microcontroller, is coupled to the first fan 112, the second fan 124 and the actuator 128 to regulate the temperature of the second container 114 such that it is within a predetermined temperature range.

In an exemplary implementation, when the thermal material is a cooling material, the first temperature and the second temperature can both be above the predetermined range of temperature.

In another exemplary implementation, when the thermal material is a heating material, the first temperature and the second temperature can both be below the predetermined range of temperature.

In another embodiment, the rate of temperature change, as registered by T1 118, is generally slower than T2 120 since the temperature change effected in the second container 114 is mostly only through conduction through the walls of the second container 114. Using readings from both T1 118 and T2 120, a correlation can be arrived at between the two, i.e., for a required temperature within the second container 114, the temperature of the second chamber 116 can be arrived at.

In one exemplary implementation, when the temperature of the second chamber 116 is cooled to within the predetermined range, the microcontroller turns the first fan 112 OFF. The first chamber 110 and the second chamber 116 are made of a thermally insulating material such as dense polystyrene, in order to minimise thermal leakage. In the event that the temperature of the second chamber 116 is cooled to below the predetermined range, the microcontroller operates the actuator 128 to open the valve 126, and the second fan 124. The air at the second temperature is forced into the second chamber 116 and the temperature of the second chamber 116 rises. Once the temperature is within the predetermined range, the valve 126 is closed and the second fan 124 is turned OFF.

In another exemplary implementation, when the temperature of the second chamber 116 is heated to within the predetermined range, the microcontroller turns the first fan 112 OFF. The first chamber 110 and the second chamber 116 are made of a thermally insulating material such as dense polystyrene, in order to minimise thermal leakage. In the event that the temperature of the second chamber 116 is heated to above the predetermined range, the microcontroller operates the actuator 128 to open the valve 126, and the second fan 124. The air at the second temperature is forced into the second chamber 116 and the temperature of the second chamber 116 drops. Once the temperature is within the predetermined range, the valve 126 is closed and the second fan 124 is turned OFF.

FIG. 3 illustrates a schematic representation of an exemplary valve for the second vent of the proposed apparatus, in accordance with an embodiment of the present disclosure. In an embodiment, the actuator 128 can be a servo motor and when it operates to open the valve 126, the second fan 124 forces air at the second temperature into the second chamber 116 thereby regulating the temperature of the second chamber 116. Once the temperature is within the predetermined range, the valve 126 is closed and the second fan 124 is turned OFF.

FIGS. 4A and 4B illustrate schematic representations of the housing of the proposed apparatus, in accordance with an embodiment of the present disclosure. In an embodiment, the walls of the housing 102 can comprise compressible lattice structures 402 that can deform to absorb mechanical shocks. The housing 102 can also be provided with a set of springs 404 to aid the lattice structures in withstanding greater magnitudes of mechanical shocks.

In another embodiment, the walls of the housing 102 can comprise one or more layers of material, the materials selected from any of a composite material layer 406, an elastomer layer 408, and an insulating layer 410. The composite material layer 406 provides strength and rigidity to the housing 102, the elastomer layer 408 provides additional cushioning against shocks and the insulating layer 410 provides thermal isolation of the inside of the housing 102.

FIG. 5 illustrates an exemplary representation of the proposed apparatus with a display and control panel, in accordance with an embodiment of the present disclosure. In an embodiment, a screen 502 is configured to display the different parameters associated with the working of the apparatus including set temperature range, current temperature, working of the first fan and the second fan, the working of the servo motor operated valve, available quantity of thermal material etc.

In another embodiment, the apparatus 100 can further have a control panel 504 that can be used to input working parameters of the apparatus 100 such as temperature range to be maintained.

FIG. 6 illustrates an exemplary representation of a microcontroller-based system to operate the proposed refrigeration apparatus, in accordance with an embodiment of the present disclosure. In an embodiment, the controller can be a proportional—integral—differential (PID) control coupled with ON-OFF systems to regulate temperature within the second container 114. The speed of the first fan 112 is taken as an output of the PID control with gain values of K_{P} (proportionality constant), K_{I} (integral constant) and K_{D} (derivative constant) while using the value of temperature as sensed by T2 120 in feedback.

In an embodiment, input from T2 120 serves to control the speed of the first fan 112, and the input from T1 118 serves to control the operation of the actuator 128 to operate the valve 126 and the speed of the second fan 124. A log of the operating data can be logged on to a memory card that is operatively coupled to the microcontroller.

In another embodiment, the microcontroller is also configured to register data from any other sensor provided in the apparatus 100. In an exemplary embodiment, other sensors can include pH meters, pressure sensors etc. The microcontroller is configured to display the above parameters on the provided screen.

In another embodiment, the apparatus can be powered by any suitable energy storage device such as a lithium-ion based battery.

In an exemplary implementation, the proposed apparatus 100 can be a refrigeration apparatus configured for organ transport such as heart transport. The following section describes the particular implementation of the proposed apparatus to maintain temperature of an object. It can be appreciated by those skilled in the art that the embodiments described hereunder is only one implementation of the proposed apparatus and is used purely to illustrate the working of the proposed apparatus and may not be construed as a limitation to the scope of the proposed apparatus.

In an aspect, the proposed apparatus can be a refrigeration apparatus that can maintain a temperature inside a container between 2° C. and 8° C. This is made possible using dry ice, with the temperature inside the container capable of decreasing from about 25° C. to under 8° C. within 6 minutes. The proposed refrigeration apparatus can be configured for organ transport such as heart transport. The apparatus can, further, be adapted to be mounted onto an unmanned aerial vehicle (UAV) such as a drone aircraft. In an implementation, the apparatus, without the heart weighs around 3.4 kg, and with it, around 3.7 kg. In comparison with similar systems known in the art, the proposed apparatus is capable of achieving a preservation time of about 6 hours while consuming about 1086 mAh of energy, which is about 91% reduction in energy consumption and about 65% reduction in system weight.

FIG. 7 illustrates an exemplary representation of a multi-function refrigeration apparatus, in accordance with an embodiment of the present disclosure. In an embodiment, the apparatus 700 comprises a housing 702 with a first container 704 (hereinafter, also referred to as “refrigerant container”) placed in a first chamber 706. The refrigerant container 704 is configured to hold a quantity of a refrigerant (not shown in figure). In an exemplary embodiment, the refrigerant can be dry ice. In another embodiment, the refrigerant container 704 is coupled with a first fan (not shown in figure) that induces air at a first temperature to flow through the refrigerant container 704 in order that the air is cooled by the dry ice. In another embodiment, the refrigerant container 704 can be provided with one or more vents (not shown in figure) to allow outflow of cooler air.

In an exemplary implementation, the air at the first temperature can be from the ambient or from another chamber of the one or more chambers.

In another embodiment, a second container 708 (hereinafter, also referred to as “heart container”) is placed in a second chamber 710. The heart container 708 can be made of a thermally conducting material such as stainless steel and can be configured to hold an item to be cooled, such that once the heart container 708 is closed, the item to be cooled in fully isolated from the ambient.

In another embodiment, the second chamber 710 can be thermally coupled to the first chamber 706. The thermal coupling can be in the form of one or more vents between the first chamber 706 and the second chamber 710 corresponding to the one or more vents in the refrigerant container 704. In another embodiment, the housing 702 can be sealed by a lid so as to thermally isolate the inside of the housing 702 from the ambient.

In another embodiment, the second chamber 710 is equipped with a temperature sensor T1 712 to detect temperature within the second chamber 710. The heart container 708 can also be equipped with a temperature sensor T2 714 to detect temperature in the heart container 708. The temperatures from either or both sensors can be used to monitor and regulate the temperature inside the second chamber 710 and the heart container 708.

In another embodiment, the first chamber 706 and the second chamber 710 are made of a thermally insulating material such as dense polystyrene, to minimise thermal leakage.

In another embodiment, the second chamber 710 also comprises an opening to a source of air at a second temperature that is normally closed by a valve 716 that can be operated by any suitable actuator such as a servo motor. The opening can be provided with a second fan to induce air flow into the second chamber 710.

In another embodiment, the first fan forces air at the first temperature into the refrigerant container 704 and over the dry ice, thereby cooling the air. The cooled air is then forced into the second chamber 710, thereby lowering the temperature of the second chamber 710, and consequently, of the heart container 708.

In another embodiment, temperatures of the second chamber 710 and the heart container 708 are detected by the sensors T1 712 and T2 714 respectively. The temperatures can be fed to a control unit which is configured to monitor the temperature. The control unit, which can be a microcontroller, can be coupled to the first fan, the second fan and the valve 716 to regulate the temperature of the heart container 708 such that it is within a predetermined temperature range.

In an exemplary implementation, the first temperature and the second temperature can both be above the predetermined range of temperature.

In another embodiment, the temperature changes as registered by T2 714 is generally slower than T1 712 since the temperature change effected in the heart container 708 is mostly through conduction through the walls of the heart container 708. Using readings from both T1 712 and T2 714, a correlation can be arrived at between the two, i.e., for a required temperature within the heart container 708, the temperature of the second chamber 710 can be arrived at.

In another embodiment, when the temperature of the second chamber 710 is cooled to within the predetermined range, the microcontroller turns the first fan OFF to arrest the flow of cooled air into the second chamber 710. In the event that the temperature of the second chamber 710 is cooled to below the predetermined range, the microcontroller operates the valve 716 to open it and allow inflow of air at the second temperature into the second chamber 710 thereby raising the temperature of the second chamber 710. Once the temperature is within the predetermined range, the valve 716 is closed. This cycle of heating and cooling is carried out to maintain the temperature of the heart container 708 within the predetermined range. This cycle can be continued until the dry ice has fully sublimated.

In another embodiment, the heart container 708 is adapted to hold a biological sample, which can be an organ for transport. In the present exemplary embodiment, the organ being transported can be a heart.

In another embodiment, the second chamber 710 can further comprise a reservoir 718 configured to hold a preservation solution such as a cardioplegic solution, for the heart that limits the degradation of the heart during transport. A pulsatile pump 720 (such as a peristaltic pump) fluidically coupled with the reservoir 718 and the heart container 708 is configured to pump the preservation solution to the heart container 708. Since the reservoir 718 is provided within the second chamber 710, the temperature of the preservation solution is also maintained low, which is desirable.

In another embodiment, the heart container 708 can further be coupled to a second reservoir 722 where excess preservation fluid can be drained to. In an exemplary embodiment, the second reservoir 722 can be part of a recirculation set up to recirculate the preservation fluid into the heart chamber 708. In another embodiment, a pH sensor 724 can be provided downstream to the heart container 708 to measure the pH of the draining preservation fluid. The pH of the preservation fluid can provide key indications to the health of the heart being transported.

In another embodiment, the heart container 708 can be further provided with an active gimbal 726 which serves to monitor and negate any mechanical shocks experienced by the heart container 708 during transport.

In another embodiment, walls of the housing 702 can be configured to have internal lattice structures to enable better thermal insulation and to reduce weight.

FIG. 8 illustrates an exemplary representation of the refrigerant container in the proposed multi-function refrigeration apparatus, in accordance with an embodiment of the present disclosure. In an embodiment, the refrigerant container 704 is configured to hold a quantity of a refrigerant 802. In an exemplary embodiment, the refrigerant can be dry ice. In another embodiment, the refrigerant container 704 is coupled with a first fan 804 that induces ambient air to flow through the refrigerant container 704 in order that the ambient air is cooled by the dry ice 802. In another embodiment, the refrigerant container can be provided with one or more vents 806 to allow outflow of cooled air.

FIG. 9 illustrates an exemplary representation of the heart container in the proposed multi-function refrigeration apparatus, in accordance with an embodiment of the present disclosure. In another embodiment, the heart container 708 is adapted to hold a biological sample, which can be an organ for transport. In the present exemplary embodiment, the organ being transported can be a heart 902.

In an embodiment, the heart container 708 can comprise one or more fins 904 on the outside of the container to better facilitate exchange of heat between the inside and the outside of the heart container 708.

In another embodiment, the pulsatile pump 720 fluidically coupled with the reservoir 718 and the heart container 708 is configured to pump the preservation solution to the heart container 708. The flow of the preservation solution is monitored through a pressure sensor 906 provided downstream of the pump 720. Further, the preservation solution passes through a bubble trap 908 to remove any air bubbles in the stream of preservation solution.

In another embodiment, the heart container 708 can further be coupled to a second reservoir 722 where excess preservation fluid can be drained to. In an exemplary embodiment, the second reservoir 722 can be part of a recirculation set up to recirculate the preservation fluid into the heart chamber 708. In another embodiment, a pH sensor 724 can be provided downstream to the heart container 708 to measure the pH of the draining preservation fluid. The pH of the preservation fluid can provide key indications to the health of the heart 902 being transported.

In another embodiment, the pH of the preservation fluid can be sent to the microcontroller as input, based on which, the microcontroller can operate the pump 720 to either increase or decrease the flow of preservation fluid to the heart 902, hence forming a closed control loop.

In another embodiment, the apparatus can be equipped with a GPS tracker to track the transport of the organ in real time.

FIG. 10 illustrates an exemplary plot of temperatures of second chamber and heart container with time for the proposed multi-function refrigeration apparatus, in accordance with an embodiment of the present disclosure. The proposed apparatus can, in one exemplary implementation, maintain a temperature inside the heart container 708 of between 2° C. and 8° C. This is made possible using about 240 g of dry ice and the temperature inside the heart container 708 decreases from about 25° C. to under 8° C. in under 6 minutes. The threshold temperature below which the servomotor 128 operates to open the valve 126 is about 5° C.

The apparatus can, further, be adapted to be mounted onto a drone vehicle such as a drone aircraft. The apparatus, without the heart weighs around 3.4 kg, and with it, around 3.7 kg. In comparison with similar systems known in the art, the proposed apparatus is capable of achieving a preservation time of about 6 hours while consuming about 1086 mAh of energy, which is about 91% reduction in energy consumption and about 65% reduction in system weight.

It should be apparent to those skilled in the art that many more modifications besides those already described are possible without departing from the inventive concepts herein. The inventive patient matter, therefore, is not to be restricted except in the spirit of the appended claims. Moreover, in interpreting both the specification and the claims, all terms should be interpreted in the broadest possible manner consistent with the context. In particular, the terms “includes” and “including” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc. The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practised with modification within the spirit and scope of the appended claims.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### ADVANTAGES

The present disclosure provides a portable apparatus to maintain temperature of an object.

The present disclosure provides an apparatus that can maintain temperature of an object within a predetermined range.

The present disclosure provides an apparatus that is energy efficient.

The present disclosure provides a refrigeration and preservation apparatus for organ transport.

The present disclosure provides an apparatus with structural integrity to withstand nominal loads due to impact.

The present disclosure provides an apparatus with optimized design with respect to weight, size and dimensions.

We claim:

1. An apparatus to maintain temperature of a biological object, said apparatus comprising:
a thermally insulated housing comprising:
at least one first chamber configured to hold a first container, said first container adapted to hold a refrigerant;
a second chamber thermally coupled to the at least one first chamber and adapted to hold a second container, said second container atmospherically isolated from the second chamber;
a first fan coupled with the at least one first container and configured to induce flow of air at a first temperature over the refrigerant and into the second chamber, said first temperature being higher than a predetermined range of temperature for the biological object;
a valve provided in the second chamber and operable to open to a source of air at a second temperature, said second temperature being higher than the predetermined range of temperature for the biological object; and
a control unit configured to sense temperature inside the second container from one or more first temperature sensors provided inside the second container, wherein the control unit is configured to operate any of the first fan and the valve to enable the predetermined temperature range to be maintained inside the second container.

2. The apparatus as claimed in claim 1, wherein the refrigerant is dry ice.

3. The apparatus as claimed in claim 1, wherein one or more second temperature sensors are provided in the second chamber, wherein the control unit receives temperature input from the one or more second temperature sensors along with temperature input from the one or more first sensors to enable determination of correlation between temperature in the second chamber and the temperature inside the second container.

4. The apparatus as claimed in claim 1, wherein the control unit comprises a microcontroller configured to receive input from the one or more first sensors and the one or more second sensors and operate the first fan, the valve and the second fan to enable the predetermined temperature range to be maintained inside the second container.

5. The apparatus as claimed in claim 1, wherein a reservoir configured to store a preservation solution is provided in the second chamber, and wherein a pump is configured to circulate the preservation solution to the biological object.

6. The apparatus as claimed in claim 5, wherein a pressure sensor is configured to measure flow pressure of the preservation solution, and wherein the measured flow pressure value is provided as input to the control unit to enable the control unit to operate the pump to provide flow of the preservation solution that is within a predetermined range of pressure.

7. The apparatus as claimed in claim 5, wherein a pH sensor is provided at a location so as to enable the sensor to measure pH of the preservation solution after the preservation solution has interacted with the biological object, and wherein the measured pH is provided as input to the control unit to enable the control unit to operate the pump to provide optimal flow of the preservation solution based on the measured pH of the used preservation solution.

8. The apparatus as claimed in claim 1 wherein a drain is provided to receive used preservation solution.

9. The apparatus as claimed in claim 1, wherein a second fan is provided to induce flow of ambient through the valve when the valve is open.

10. The apparatus as claimed in claim 1, wherein the second container is made of a thermally conducting material.

11. The apparatus as claimed in claim 1, wherein the walls of the housing comprise one or more elements configured to absorb physical shock, said one or more elements selected from a group comprising springs, lattice structures and elastomer layers.

12. The apparatus as claimed in claim 1, wherein the apparatus is provided with a control unit that is configurable.

13. An apparatus to maintain temperature of an object, said apparatus comprising:
a thermally insulated housing comprising:
at least one first chamber configured to hold a first container, said first container adapted to hold a thermal material;
a second chamber thermally coupled to the at least one first chamber and adapted to hold a second container, said second container atmospherically isolated from the second chamber;
a first fan coupled with the at least one first container and configured to induce flow of air at a first temperature over the thermal material and into the second chamber, said first temperature being outside of a first limit of a predetermined range of temperature for the biological object;
a valve provided in the second chamber and operable to open to a source of air at a second temperature, said second temperature being outside of the first limit of the predetermined range of temperature for the biological object; and
a control unit configured to sense temperature inside the second container from one or more first temperature sensors provided inside the second container, wherein the control unit is configured to operate any of the first fan and the valve to enable the predetermined temperature range to be maintained inside the second container.

14. The apparatus as claimed in claim 13, wherein the thermal material is a refrigerant.

15. The apparatus as claimed in claim 13, wherein the thermal material is a heating material.

16. The apparatus as claimed in claim 13, wherein one or more second temperature sensors are provided in the second chamber, wherein the control unit receives temperature input from the one or more second temperature sensors along with temperature input from the one or more first sensors to enable determination of correlation between temperature in the second chamber and the temperature inside the second container.

17. The apparatus as claimed in claim 13, wherein the control unit comprises a microcontroller configured to receive input from the one or more first sensors and the one or more second sensors and operate the first fan, the valve and the second fan to enable the predetermined temperature range to be maintained inside the second container.
